(19) United States
(12) Patent Application Publication
(10) Pub. No.: US 2023/0165722 A1
(43) Pub. Date: Jun. 1, 2023

(54) AUDIO DEVICE OUTPUT ENERGY CONTROL METHOD FOR PROTECTING HEARING

(71) Applicant: SHENZHEN DASHI SCIENCE AND TECHNOLOGY CO., LTD., Shenzhen (CN)

(72) Inventor: XinYu Li, Shenzhen (CN)

(21) Appl. No.: 17/544,951

(22) Filed: Dec. 8, 2021

(30) Foreign Application Priority Data
Nov. 29, 2021 (CN) 202111432394.4

(51) Int. Cl. A61F 11/14; A61F 11/08

(52) U.S. Cl. CPC A61F 11/145; A61F 11/08

(57) ABSTRACT

The present invention discloses an audio device output energy control method for protecting hearing, the method including: step S1, obtaining energy or pressure at a speaker or at any point in a sound field from the speaker to a tympanic membrane by means of calculation or measurement, which serves as the energy or pressure at the tympanic membrane or cochlea of a user after compensation or calculation; step S2, calculating a cumulative hearing loss value according to the energy or pressure at the tympanic membrane or cochlea of the user, or calculating cumulative energy at any point in the sound field from the speaker to the tympanic membrane according to the energy or pressure at that location, and then equivalently calculating a cumulative hearing loss value at the tympanic membrane or cochlea of the user; step S3, comparing the cumulative hearing loss value with a preset hearing loss threshold, and performing step S4 if the cumulative hearing loss value reaches the hearing loss threshold; and step S4, reducing a volume of the audio device. The present invention can monitor the energy at the tympanic membrane or cochlea of the user in real time and adjust the output energy of the audio device in time, thus realizing hearing protection.

### CROSS REFERENCE TO RELATED APPLICATION

This application claims priority benefit of Chinese Application No. 202111432394.4, filed on Nov. 29, 2021, and the entire contents of which are incorporated herein by reference.

### TECHNICAL FIELD

The present invention relates to an audio device output energy control method, and in particular to an audio device output energy control method for protecting hearing.

### BACKGROUND

As people are using audio devices such as headphones more and more frequently, hearing protection has become quite an important matter, and in many cases, people realize the need to protect their hearing only after a hearing problem has been noticed. However, in most cases, hearing loss is largely irreversible. For young people who often wear headphones, the leading cause of hearing loss is exposure to loud sound. According to available statistics, when iPhone 6 in-ear earphones are played at 5-6 levels of volume for 8 hours or at 7-8 levels of volume for 1 hour, the hearing safety threshold will be reached, and once this hearing threshold is exceeded, temporary hearing loss will be caused, while long-time exposure may cause irreversible hearing loss.

In order to solve the problem of hearing loss, in the related art, ambient noise can be reduced through Active Noise Cancellation (ANC), so that users can listen to music at a lower volume, thus achieving the purpose of hearing protection. However, if a user unconsciously increases the volume, his/her hearing may be impaired as well. In addition, the related art may also provide simple alerts through application programs (Apps), which, however, can hardly work effectively and cannot really achieve the purpose of preventing hearing loss.

### SUMMARY

The technical, problem to be solved by the present invention is to, in view of the shortcomings of the related art, provide an audio device output energy control method that can monitor the energy at the tympanic membrane or cochlea of the user in real time and adjust the output energy of the audio device in time, thus achieving hearing protection without affecting the user's use experience.

To solve the above technical problem, the present invention adopts the following technical solution.

An audio device output energy control method for protecting hearing, including the following steps: step S1, obtaining energy or pressure at a speaker or at any point, in, a sound field from the speaker to a tympanic membrane by means of calculation or measurement, which serves as the energy or pressure at the tympanic membrane or cochlea of a user after compensation or calculation; step S2, calculating a cumulative hearing loss value according to the energy or pressure at the tympanic membrane or cochlea of the user, or calculating cumulative energy at any point in the sound field from the speaker to the tympanic membrane according to the energy or pressure at that location, and then equivalently calculating a cumulative hearing loss value at the tympanic membrane or cochlea of the user; step S3, comparing the cumulative hearing loss value with a preset hearing loss threshold, and performing step S4 if the cumulative hearing loss value reaches the hearing loss threshold; and step S4, reducing a volume of the audio device.

Optionally, in the step S1, the energy or pressure at the tympanic membrane or cochlea of the user is obtained by calculating an audio stream transmitted to the speaker of the audio device.

Optionally, in the step S1, the audio stream is subject to calculation and compensation using an A-weighted network. Starting from the A-weighted transfer function, the transfer function of the above A-weighted simulation system is converted into a digital filter using a bilinear transformation method and a filter coefficient h(n) is obtained; let input audio data be x(n), then an output processed by the A-weighted network is y(n); and the y(n) data processed by the A-weighted network is, subject to sound pressure level calculation using a sound pressure level calculation formula to obtain an A-weighted sound pressure level value of the current audio stream,

Optionally, according to the A-weighted sound pressure level of the current audio stream as well as the sound pressure level compensation value at the tympanic membrane of the user measured at the laboratory, a compensation calculation is performed to obtain the sound pressure level value at the tympanic membrane of the user.

Optionally, in the step S1, the sound pressure level near the speaker is obtained through measurement of a microphone of the audio device, and the sound pressure level near the speaker is compensated to obtain the sound pressure level at the tympanic membrane.

Optionally, in the step S1, according to the A-weighted sound pressure level value of the current audio stream, and the transfer function from a test point to the tympanic membrane of the user, the sound pressure level at the tympanic membrane of the user is obtained through calculation.

Optionally, the method further includes a hearing loss protection triggering step in which: the system performs a protection action by means of the cumulative hearing loss value and initiates the protection action when the cumulative hearing loss value reaches the preset hearing loss threshold.

Optionally, a plurality of preset hearing loss thresholds are included, each of which corresponds to a different degree of hearing loss urgency.

Optionally, a hearing protection function is turned on or off via a preset App, and the steps S1 to S4 are not performed when the hearing protection function is turned off.

In the audio device output energy control method for protecting hearing disclosed in the present invention, firstly, the amount of energy at the tympanic membrane or cochlea of the user is monitored in real time by means of calculation or measurement, and the current cumulative hearing loss amount is calculated, and when the cumulative hearing loss amount, exceeds a preset, threshold, the system reduces the output energy. At the same time, when the rate of increase in the cumulative hearing loss amount is monitored to increase, the protection of the system becomes stronger, and when the rate of increase in the cumulative hearing loss amount is monitored to decrease, the protection of the system becomes gentler. It can be concluded that no matter how the user adjusts the output energy, the cumulative hearing loss amount will not exceed the preset threshold. Based on the above principle, it can be seen that the present invention can not only monitor the sound pressure level in the tympanic membrane of the user in real time and adjust the output energy in time, but also achieve hearing protection without affecting the user's use experience, and thus can well meet the application requirements.

### BRIEF DESCRIPTION OF DRAWINGS

FIG. 1 is a flowchart of an audio device output energy control method for protecting hearing according to the present invention;

FIG. 2 is the graph of the frequency domain curves of an A-weighted network; and

FIG. 3 is a schematic diagram of the structure of the A-weighted network.

### DETAILED DESCRIPTION

The present invention will be described in more detail below in conjunction with the accompanying drawings and embodiments.

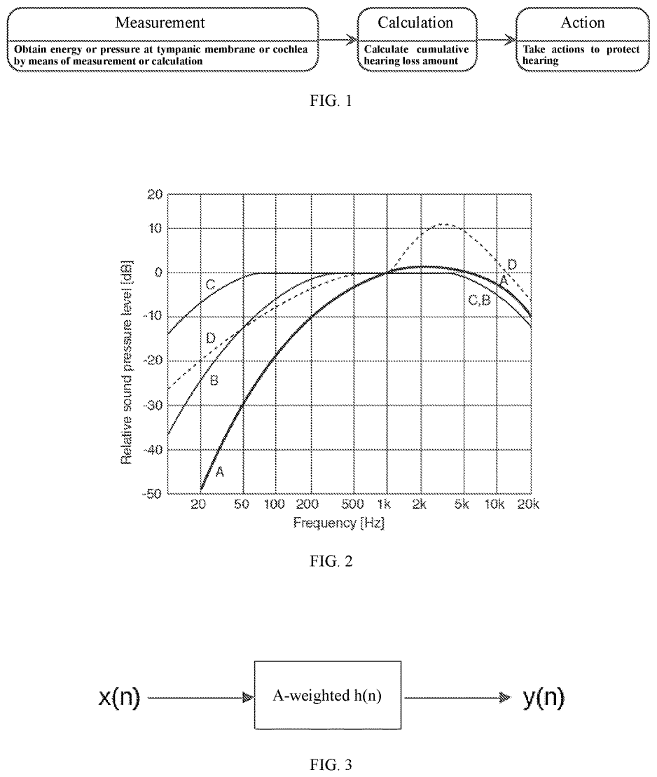

The present invention discloses an audio device output energy control method for protecting hearing which, with reference to FIG. 1, includes the following steps:

step S1, obtaining energy or pressure at a speaker or at any point in a sound field from the speaker to a tympanic membrane by means of calculation or measurement, which serves as the energy or pressure at the tympanic membrane or cochlea, of a user after compensation or calculation, where, specifically, the energy or pressure is not limited to sound pressure, sound pressure level, sound power, sound intensity or sound intensity level, etc.;

step S2, calculating a cumulative hearing loss value according to the energy or pressure at the tympanic membrane or cochlea of the user, or calculating cumulative energy at any point in the sound field from the speaker to the tympanic membrane according to the energy or pressure at that location, and then equivalently calculating a cumulative hearing loss value at the tympanic membrane or cochlea of the user;

step S3, comparing the cumulative hearing loss value with a preset hearing loss threshold, and performing step S4 if the cumulative hearing loss value reaches the hearing loss threshold; and

step S4, reducing the volume of the audio device.

In the above method, firstly, the amount of energy at the tympanic membrane or cochlea of the user is monitored in real time by means of calculation or measurement, and the current cumulative hearing loss amount is calculated, and when the cumulative hearing loss amount exceeds a preset threshold, the system reduces the output energy. At the same time, when the rate of increase in the cumulative hearing loss amount is monitored to increase, the protection of the system becomes stronger, and when the rate of increase in the cumulative hearing loss amount is monitored to decrease, the protection of the system becomes gentler. It can be concluded that no matter how the user adjusts the output energy, the cumulative hearing loss amount will not exceed the preset threshold. Based on the above principle, it can be seen that the present invention can not only monitor the sound pressure level in the tympanic membrane of the user in real time and adjust the output energy in time, but also achieve hearing protection without affecting the user's use experience, and thus can well meet the application requirements.

According to the present invention, there are the following two approaches to acquiring the sound pressure at the tympanic membrane of the user.

Approach I:

In the step S1, the energy or pressure at the tympanic membrane or cochlea of the user is obtained by calculating an audio stream transmitted to the speaker of the audio device.
A-Weighted is a standard weighting curve for audio measurements, which is used for reflecting the response characteristics of human ears. The sound pressure level is derived from A-weighted and is expressed as dBA. A-weighted is widely used in the measurement of noise and stable audio signals, and its frequency domain curves are as shown in FIG. 2. It can be seen from FIG. 2 that when using A-weighted for audio measurement, the weighting for low frequencies will be lower than that for middle and high frequencies. Since A-weighted is the most meaningful for describing the frequency response of human ear hearing relative to real acoustics, it is the most widely used.

To this end, in this embodiment, in the step S1, according to the national standard GB/T 3785.1-2010, the audio stream is subject to calculation and compensation (the compensation value is determined by the difference between the sound pressure at the speaker and the sound pressure at the tympanic membrane measured in the laboratory) using the A-weighted network, the transfer function of the A-weighted simulation system being as follows:

$$A ( S ) = 1 {0}^{\frac{- {A}_{1000}}{20}} [ \frac{{Ω}_{4}^{2} {S}^{2}}{{( {S}^{2} + {Ω}_{1}^{2} )}^{2} ( {S}^{2} + {Ω}_{2}^{2} ) ( {S}^{2} + {Ω}_{3}^{2} ) {( {S}^{2} + {Ω}_{4}^{2} )}^{2}} ] ;$$

where:

Ω₁=2πf₁,Ω2=2πf₂,Ω₃=2πf₃,Ω₄=2πf₄,A₁₀₀₀=1.9997

f₁=20.60 Hz,f₂1077 Hz,f₃=737.9 Hz,f₄=12194 Hz

then the transfer function of the above A-weighted simulation system is converted into a digital filter using a bilinear transformation method, and a filter coefficient h(n) is obtained;

specifically, in the above step, the transfer function of the A-weighted simulation system is converted into a digital filter using a bilinear transformation method based on MATLAB;

with reference to FIG. 3, let input audio data be x(n), then an output processed by the A-weighted network is y(n); and

the y(n) data processed by the A-weighted network is subject to sound pressure level calculation using a sound pressure level calculation formula to obtain an A-weighted sound pressure value (dBA value) of the current audio stream,

the sound pressure level calculation formula being:

$$S P L = 20 \times {log}_{10} \frac{{p}_{e}}{{P}_{r e f}} ;$$

where

$${p}_{e} = \sqrt{\frac{1}{N} {\sum }_{n = 1}^{N} {x}^{2} ( n )} , {p}_{ref = 2 \times {10}^{- 5}} :$$

In this embodiment, the audio device system is tested with pink noise signals and the relationship between dBA values and volume levels is obtained, as shown in the following table:

| | A-weighted | Measured | |
|---|---|---|---|
| | sound | sound | |
| | pressure level | pressure | Difference |
| Volume level | (dBA) | (dBA) | (dBA) |
| 10 | 45 | 78.9 | 33.9 |
| 11 | 49 | 82.5 | 33.5 |
| 12 | 53 | 86.7 | 33.7 |
| 13 | 56 | 89.7 | 33.7 |
| 14 | 59 | 92.9 | 33.9 |
| 15 | 62 | 95.5 | 33.5 |
| 16 | 65 | 99 | 34 |

From the above table, it can be seen that there is a fixed difference of 33.7 between the measured sound pressures and the calculated sound pressures. Therefore, the real sound pressure may be obtained by adding the compensation value of 33.7 dBA to the A-weighted calculation result. It should be noted that the difference of 33.7 here is related to specific products, and the compensation values measured for different products are different.

Approach II:

In the step S1, energy in a sound field of the speaker is obtained through measurement of a microphone of the audio device, and the energy in the sound field of the speaker is compensated to obtain the energy at the tympanic membrane or at the cochlea.

For example, when the audio device is an in-ear noise-reduction headphone, it has three MICs, namely FE FB and Talk MICs, where the FB MIC is located near the speaker at a certain distance from the tympanic membrane. The data obtained via the FB MIC may be compensated as the sound pressure in the tympanic membrane. In addition, the energy is transmitted to the tympanic membrane through the speaker, and the energy decreases in a gradient from the speaker to the tympanic membrane, and the value of the gradient may be measured with laboratory data. The sound pressure at the tympanic membrane is the compensated sound pressure at the FB MIC. Specifically, the FB MIC data is subject to the A-weighted network calculation and then compensated to obtain the sound pressure at the tympanic membrane.

Regarding the calculation of the cumulative hearing loss, in this embodiment, according to the results of relevant researches on hearing impairment (with reference to the WHO standards), it is found that hearing loss is related to two factors: loudness and duration, and the relationship between time and loudness is not linear, as shown in the following table:

| Sound | Duration |
|---|---|
| pressure (dBA) | (min) |
| 80 | 480 |
| 83 | 240 |
| 86 | 120 |
| 89 | 60 |
| 92 | 30 |
| 95 | 15 |
| 98 | 7.5 |
| 101 | 3.75 |

On this basis, in the step S2, the cumulative hearing loss value is calculated based on the following 80 dBA±6 dBA normalized cumulative hearing loss formula:

tₜₒₜₐₗ=t₈₀+2t₈₃+4*t₈₆+8*t₈₉+16t₉₂+32*t₉₅+64*t₉₈+128*t₁₀₁−K*tₙₒᵣₘₐₗ

where t₈₀ denotes the cumulative time for the sound pressure of 80 dBA±6 dBA, t₈₃ denotes the cumulative time for the sound pressure of 83 dBA±6 dBA, and so on, tₙₒᵣₘₐₗ denotes the cumulative time for the sound pressure below 80 dBA±6 dBA, and K denotes a recovery coefficient, and according to user habits, the value of K is 1.

In practical applications, when the calculated tₜₒₜₐₗ is less than 480, hearing loss will not occur; and when tₜₒₜₐₗ is greater than 480, temporary hearing loss will occur, which can be recovered by ear rest.

When the system is turned off, the user's idle time is not measurable, so the maximum possible estimation can be adopted, that is, the user's ears, have rested for half an hour by default.

With regard to the triggering of hearing loss protection, in the step S2 of this embodiment, an average energy value of the tympanic membrane or cochlea per second is calculated, and substituted into the hearing loss formula to obtain tₜₒₜₐₗ.

The specific steps regarding hearing loss protection are as follows:

| | Protection action in case the |
|---|---|
| Cumulative hearing loss tₜₒₜₐₗ | cumulative hearing loss increases |
| Greater than 240 | Decrease the volume by 1 level |
| Greater than 360 | Decrease the volume by 1 level |
| Greater than 420 | Decrease the volume by 1 level |
| Greater than 450 | Decrease the volume by 1 level |
| Greater than 465 | Decrease the volume by 1 level |
| Greater than 480 | Control the overall volume of the |
| | system not to exceed 80 dBA ± 6 dBA |
| | Recovery action in case the |
| Cumulative hearing loss tₜₒₜₐₗ | cumulative hearing loss decreases |
| Less than 480 | Increase the volume by 1 level |
| Less than 465 | Increase the volume by 1 level |
| Less than 450 | Increase the volume by 1 level |
| Less than 420 | Increase the volume by 1 level |
| Less than 360 | Increase the volume by 1 level |
| Less than 240 | Restore the normal volume |

The present invention optionally sets a hearing loss protection triggering step in which: the system performs a protection action by means of the cumulative hearing loss value and initiates the protection action when the cumulative hearing loss value reaches the preset hearing loss threshold. In addition, a plurality of preset hearing loss thresholds are, further included, each of which corresponds to a different degree of hearing loss urgency.

In the specific operation process, the hearing loss factor and the human ear recovery factor jointly work on the cumulative hearing loss variable to obtain a total cumulative hearing loss variable of the system. The system performs the protection action by means of the cumulative hearing loss variable, and when the cumulative hearing loss variable reaches the preset threshold, the protection action is initiated. There are several preset thresholds, each of which corresponds to a different degree of hearing loss urgency and a different strength of protection action. Through this method, it can be learned that no matter how the user adjusts the volume, the system can effectively control the cumulative hearing loss within the specified threshold to protect hearing.

This embodiment also involves the steps of turning on and off the hearing loss protection function which specifically refer to turning on or off the hearing protection function via a preset App, and the steps S1 to S4 are not performed when the hearing protection function is turned off.

The above description only illustrates better embodiments of the present invention, and is not intended to limit the present invention. Any modification, equivalent replacement or improvement, etc., made within the technical scope of the present invention should be included in the scope of protection of by the present invention.

1. An audio device output energy control method for protecting hearing, comprising:
step S1, obtaining energy or pressure at a speaker or at any point in a sound field from the speaker to a tympanic membrane by means of calculation or measurement, which serves as the energy or pressure at the tympanic membrane or cochlea of a user after compensation or calculation;
step S2, calculating a cumulative hearing loss value according to the energy or pressure at the tympanic membrane or cochlea of the user, or calculating cumulative energy at any point in the sound field from the speaker to the tympanic membrane according to the energy or pressure at that location, and then equivalently calculating a cumulative hearing loss value at the tympanic membrane or cochlea of the user;
step S3, comparing the cumulative hearing loss value with a preset hearing loss threshold, and performing step S4 if the cumulative hearing loss value reaches the hearing loss threshold; and
step S4, reducing a volume of the audio device.

2. The audio device output energy control method for protecting hearing of claim 1, wherein, in the step S1, the energy or pressure at the tympanic membrane or cochlea of the user is obtained by calculating an audio stream transmitted to the speaker of the audio device.

3. The audio device output energy control method for protecting hearing of claim 2, wherein, in the step S1, the audio stream is subject to calculation and compensation using an A-weighted network, the transfer function of an A-weighted simulation system being:
$$A ( S ) = 1 {0}^{\frac{- {A}_{1000}}{20}} [ \frac{{Ω}_{4}^{2} {S}^{2}}{{( {S}^{2} + {Ω}_{1}^{2} )}^{2} ( {S}^{2} + {Ω}_{2}^{2} ) ( {S}^{2} + {Ω}_{3}^{2} ) {( {S}^{2} + {Ω}_{4}^{2} )}^{2}} ] ;$$
where:
Ω₁=2πf₁,Ω2=2πf₂,Ω₃=2πf₃,Ω₄=2πf₄,A₁₀₀₀=1.9997
f₁=20.60 Hz,f₂1077 Hz,f₃=737.9 Hz,f₄=12194 Hz
then the transfer function of the above A-weighted simulation system is converted into a digital filter using a bilinear transformation method, and a filter coefficient h(n) is obtained,
let input audio data be x(n), then an output processed by the A-weighted network is y(n), and
the y(n) data processed by the A-weighted network is subject to sound pressure level calculation using a sound pressure level calculation formula to obtain an A-weighted sound pressure value of the current audio stream,
the soundpressure level calculation formula being:
$$SPL = 20 \times {log}_{10} \frac{{p}_{e}}{{P}_{r e f}} ;$$
where
$${p}_{e} = \sqrt{\frac{1}{N} {\sum }_{n = 1}^{N} {y}^{2} ( n )} , {p}_{ref = 2 \times {10}^{- 5}} :$$

4. The audio device output energy control method for protecting hearing of claim 1, wherein, in the step S1, energy in a sound field of the speaker is obtained through measurement of a microphone of the audio device, and the energy in the sound field of the speaker is compensated to obtain the energy at the tympanic membrane or at the cochlea.

5. The audio device output energy control method, for protecting hearing of claim 1, wherein, in the step S2, the cumulative hearing loss value is calculated based on the following 80 dBA±6 dBA normalized cumulative hearing loss formula:
tₜₒₜₐₗ=t₈₀+2t₈₃+4*t₈₆+8*t₈₉+16t₉₂+32*t₉₅+64*t₉₈+128*t₁₀₁−K*tₙₒᵣₘₐₗ
where t₈₀ denotes the cumulative time for the sound pressure of 80 dBA±6 dBA, t₈₃ denotes the cumulative time for the sound pressure of 83 dBA±6 dBA, and so on, tₙₒᵣₘₐₗ denotes the cumulative time for the sound pressure below 20 dBA±6 dBA, and K denotes a recovery coefficient.

6. The audio device output energy control method for protecting hearing of claim 5, wherein the value of K is 1.

7. The audio device output energy control method for protecting hearing of claim 5, wherein, in the step S2 an average energy value of the tympanic membrane or cochlea per second is calculated, and is substituted into the hearing loss formula to obtain tₜₒₜₐₗ.

8. The audio device output energy control method for protecting hearing of claim 5, further comprising a hearing loss protection triggering step in which: the system performs a protection action by means of the cumulative hearing loss value and initiates the protection action when the cumulative hearing loss value reaches the preset hearing loss threshold.

9. The audio device output energy control method for protecting hearing of claim 8, wherein a plurality of preset hearing loss thresholds are included, each of which corresponds to a different degree of hearing loss urgency.

10. The audio device output energy control method for protecting hearing of claim 1, wherein a hearing protection function is turned on or off via a preset application program, and the steps S1 to S4 are not performed when the hearing protection function is turned off.
